(19) United States
(12) Patent Application Publication
(10) Pub. No.: US 2006/0020033 A1
(43) Pub. Date: Jan. 26, 2006

(54) Method for treating dermatological viral infections

(76) Inventor: Ali Moiin, Grosse Pointe Park, MI (US)

Correspondence Address: Brian S. Pickell;Bliss McGlynn, P.C., Suite 600, 2075 West Big Beaver Road, Troy, MI, 48084, US

(21) Appl. No.: 10/896,201

(22) Filed: Jul. 21, 2004

(51) Int. Cl. A61K 31/195; A61N 1/30

(52) U.S. Cl. 514/561; 604/20

(57) ABSTRACT

A method for treating dermatological viral infections includes applying a photosensitizing agent to virally infected skin, allowing the virally infected skin to become photosensitive, and illuminating a beam of visible light onto the photosensitive skin to produce a photodynamic reaction substantially reducing the amount of viral infection to the skin.

### BACKGROUND OF THE INVENTION

1. Field of the Invention

The present invention relates, generally, to a method for treating dermatological viral infections and, more particularly, to such a method using a light-sensitizing agent and light.

2. Description of the Related Art

Some viral infections cause non-cancerous growths or lesions to develop in top layers of the skin. The subject viruses can enter the skin through small breaks or hair follicles in the skin and can also affect internal organs. Two common such skin growths, for example, are molluscum contagiosum (molluscum) and warts.

The skin growths can appear in various shapes and sizes and be found on various parts of the body. For instance, a molluscum is usually small, flesh-colored or pink, and dome-shaped and may appear shiny and define a small indentation in the center of the top of the molluscum. Clusters of the growths are often found on the skin of the chest, abdomen, arms, groin, and/or buttocks. Such clusters are often found also on the face, in general, and eyelids, in particular. And, because they can be easily spread by skin-to-skin contact, the growths are usually found in areas of the skin that touch each other, such as in the folds of an arm or in the groin. The skin growths can become red or inflamed as well. In a person having a disease of the immune system, the growths can further be very large and located on the face.

There is an increased risk of skin growths developing in young children, especially among siblings, who may not yet have developed an immunity to a particular virus. In fact, children tend to have a higher incidence of skin growths as compared to that of adults. Nevertheless, a virus can be sexually transmitted if the corresponding growth is present in the genital area. It also is possible to acquire a virus from non-living objects. For example, molluscum may be spread among children by water in swimming pools.

In addition, climate and health conditions can affect the incidence of skin growths. For instance, development of molluscum seems to be more common in the warmth and humidity of tropical climates, and people infected with HIV are more susceptible to acquiring molluscum. In general, a person having a weakened immune system may be more susceptible to acquiring skin growths.

Many dermatologists recommend treating growths because they can easily spread from one area of the skin to another such area. Moreover, while some untreated skin growths eventually go away, others do not or do so only after an extended period of time and/or by leaving a scar. For example, it may take five years for all untreated molluscum to go away. In any event, the condition is easier to control and contain if treatment is started when there are only a few skin growths.

Many skin growths are treated in the same way. By way of example and not by way of limitation, they can be frozen with liquid nitrogen, destroyed with various acids or blistering solutions, or scraped off with a sharp instrument (curette). Skin growths can be also treated with an electric needle (electrocautery), a daily home application of a topical retinoid cream or gel, a topical immune modifier, or a topical anti-Viral medication. In addition, laser therapy has been effective in treating some skin growths, and new drugs are being developed to treat viral infections.

Although these procedures can be effective, some substantial discomfort is associated with freezing and scraping and use of the electric needle and laser therapy. Also, these procedures are often conducted on only older children and adults. And, if there are many skin growths, multiple treatment sessions may be needed until the growths are gone. It is also an option, especially with young children, not to treat and to wait for the untreated skin growths to go away. Further, using these treatments, it is always possible for a person's skin to become infected again with a particular virus.

Thus, there is a need for an effective method for treating dermatological viral infections that is not associated with any substantial discomfort. There is a need for an effective method for treating dermatological viral infections also for people of all ages. There is a need for an effective method for treating dermatological viral infections that also, if there are many skin growths, does not require multiple treatment sessions until the growths are gone. There is a need for an effective method for treating dermatological viral infections that also eliminates the need to wait for the skin growths to go away on their own. There is a need for an effective method for treating dermatological viral infections that also minimizes the possibility for a person's skin to get infected again with a particular virus.

### SUMMARY OF THE INVENTION

The invention overcomes the disadvantages in the related art in a method for treating dermatological viral infections. The method includes applying a photosensitizing agent to virally infected skin, allowing the virally infected skin to become photosensitive, and illuminating a beam of visible light onto the photosensitive skin to produce a photodynamic reaction substantially reducing the amount of viral infection to the skin.

One advantage of the method for treating dermatological viral infections of the present invention is that it is effective.

Another advantage of the method for treating dermatological viral infections of the present invention is that it is not associated with any substantial discomfort.

Another advantage of the method for treating dermatological viral infections of the present invention is that it is for people of all ages.

Another advantage of the method for treating dermatological viral infections of the present invention is that it, if there are many skin growths, does not require multiple treatment sessions until the growths are gone.

Another advantage of the method for treating dermatological viral infections of the present invention is that it eliminates the need to wait for the skin growths to go away on their own.

Another advantage of the method for treating dermatological viral infections of the present invention is that it minimizes the possibility for a person's skin to get infected again with a particular virus.

Other objects, features, and advantages of the method for treating dermatological viral infections of the present invention will be readily appreciated as the same becomes better understood while reading the subsequent description taken in conjunction with the accompanying drawings.

### BRIEF DESCRIPTION OF THE DRAWINGS

FIG. 1 is a schematic diagram depicting the steps of a first embodiment of the method for treating dermatological viral infections of the present invention.

FIG. 2 is a schematic diagram depicting the steps of a second embodiment of the method for treating dermatological viral infections of the present invention.

FIG. 3 is a schematic diagram depicting the steps of a third embodiment of the method for treating dermatological viral infections of the present invention.

FIG. 4 is a schematic diagram depicting the steps of a fourth embodiment of the method for treating dermatological viral infections of the present invention.

### DETAILED DESCRIPTION OF THE INVENTION

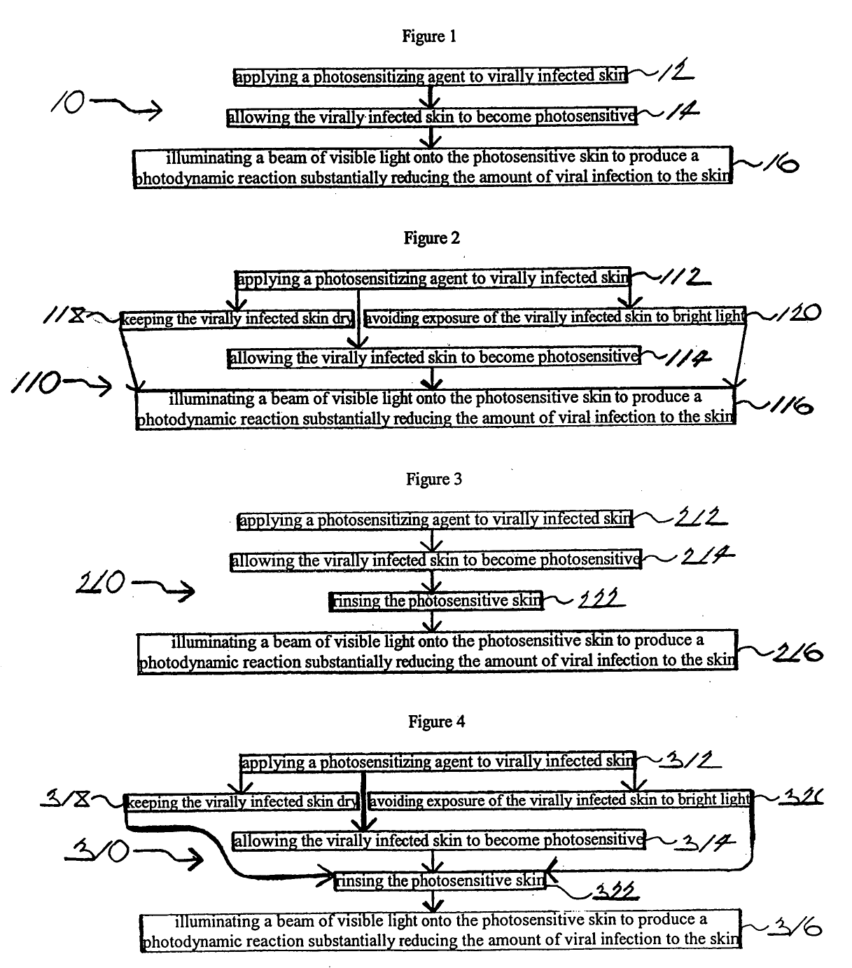

Referring now to the figures, a method for treating dermatological viral infections of the present invention is generally indicated at 10, 110, 210, and 310. More specifically, FIGS. 1 through 4 use flow diagrams to illustrate the steps in a sequence of operations of the method 10, 110, 210, 310.

Referring now to FIG. 1, the steps in a sequence of operations of a first embodiment of the method are generally indicated at 10. The method 10 includes, in general, the steps of applying a photosensitizing agent to virally infected skin 12, allowing the virally infected skin to become photosensitive 14, and illuminating a beam of visible light onto the photosensitive skin 16 to produce a photodynamic reaction substantially reducing the amount of viral infection to the skin.

In a preferred embodiment of the method 10, the dermatological virus includes warts or any virus belonging to the poxvirus family of viruses, such as molluscum contagiosum. However, it will be appreciated by those having ordinary skill in the art that the method 10 can be used on skin affected by any suitable dermatological virus. It will be appreciated also that the method can be used on any suitable affected area or volume of skin, such as an area or volume of a patient's face.

The virally infected skin to be treated is clean and dry prior to the step of applying the photosensitizing agent to the virally infected skin 12. The step of applying the photosensitizing agent 12 also is performed topically. More specifically, the photosensitizing agent is topically applied substantially directly and uniformly to only individual areas or volumes of the virally infected skin such that the photosensitizing agent is not topically applied to skin surrounding such respective areas or volumes. However, those having ordinary skill in the art will appreciate that such step can be performed other than topically.

The photosensitizing agent can further take any suitable form, such as cream, gas, gel, liquid, or paste, just to name a few. The photosensitizing agent further includes aminolevulinic acid HCL (ALA). More specifically, the photosensitizing agent is Levulan® Kerastick® Topical Solution. However, those having ordinary skill in the art will appreciate that the photosensitizing agent can be any suitable agent adapted to make the virally infected skin reactive or sensitive to visible light (photosensitive). For instance, other known photosensitizing agents include griseofulvin, thiazide diuretics, sulfonylureas, phenothiazines, sulfonamides, and tetracyclines, just to name a few.

During the time period between the step of applying the photosensitizing agent to the virally infected skin 12 and the step of exposing such skin to activating light from the beam of visible light 16, the treatment site becomes photosensitive 14. This time period is about fourteen to about eighteen hours. Those having ordinary skill in the art will appreciate, however, that any suitable time period will suffice. In any event, the treatment sites should not be washed during this time period.

The step of illuminating the beam of visible light onto the photosensitive skin 16 is performed at substantially low intensity such that the beam of visible light does not substantially heat the photosensitive skin. For example, the beam of visible light is illuminated 16 at an intensity of about 6 to about 10.9 J/cm² and, more specifically, of about 10 J/cm². The beam of visible light is illuminated 16 also for about 17 minutes and, more specifically, about 16 minutes and 40 seconds. The entire surface area or volume of the photosensitive skin lies between about two inches and about four inches from the source of the beam of visible light, which, preferably, is a laser.

However, it will be appreciated by those having ordinary skill in the art that the beam of visible light can be illuminated 16 at any suitable intensity for any suitable amount of time. It will be appreciated also that the surface area or volume of skin to be treated can lie any suitable distance from the source of the beam of visible light. It will be appreciated also that the beam of visible light can be coherent or incoherent and a continuous wave or pulsed.

Preferably, the beam of visible light is blue. More specifically, the wavelength of the beam of blue light is between about 400 nm and about 450 nm. Even more specifically, the beam of blue light is illuminated 16 using BLU-U™ Blue Light Photodynamic Therapy Illuminator. However, it will be appreciated by those having ordinary skill in the art that the beam of visible light can be any suitable color and wavelength and illuminated using any suitable source of visible light.

During the light treatment 16, the patient may experience slight discomfort in the form of tingling, stinging, prickling, and/or burning of the treated skin. However, such discomfort should lessen at the end of the treatment 16. In particular, stinging and/or burning should subside between one minute and twenty-four hours after the treatment 16. Temporarily following the treatment 16, the treated skin and, to some degree, the skin surrounding it may redden, swell, and scale as well.

Referring now to FIG. 2, where like numerals increased by 100 are used to designate operations like those of FIG. 1, the steps in a sequence of operations of a second embodiment of the method are generally indicated at 110. The method 110 includes, in general, the step of applying a photosensitizing agent to virally infected skin 112. The method 110 includes also the steps of keeping the virally infected skin dry 118 and avoiding exposure of the virally infected skin to bright light 120. The method 110 includes also the steps of allowing the virally infected skin to become photosensitive 114 and illuminating a beam of visible light onto the photosensitive skin 116 to produce a photodynamic reaction substantially reducing the amount of viral infection to the skin.

In a preferred embodiment of the method 110, the step of avoiding exposure of the photosensitive treatment sites to bright light 120 includes avoiding exposure to sunlight, prolonged or intense light, or bright, indoor light during the period prior to visible-light treatment 116. Bright indoor lights can include, but are not limited to, examination lamps, operating-room lamps, tanning beds, or lights at close proximity to the treatment sites. Otherwise, such exposure may result in a stinging and/or burning sensation and may cause erythema and/or edema of the photosensitive treatment sites.

Referring now to FIG. 3, where like numerals increased by 200 are used to designate operations like those of FIG. 1, the steps in a sequence of operations of a third embodiment of the method are generally indicated at 210. The method 210 includes, in general, the steps of applying a photosensitizing agent to virally infected skin 212, allowing the virally infected skin to become photosensitive 214, rinsing the photosensitive skin 222, and illuminating a beam of visible light onto the photosensitive skin 216 to produce a photodynamic reaction substantially reducing the amount of viral infection to the skin.

In a preferred embodiment of the method 210, the step of rinsing the photosensitive skin 222 is performed gently and with water. However, those having ordinary skill in the art will appreciate that such step can be performed with any suitable rinsing agent. The rinsed skin should be patted dry as well.

Referring now to FIG. 4, where like numerals increased by 300 are used to designate operations like those of FIG. 1, the steps in a sequence of operations of a fourth embodiment of the method are generally indicated at 310. The method 310 includes, in general, the step of applying a photosensitizing agent to a virally infected area of skin 312. The method 310 includes also the steps of keeping the virally infected area of skin dry 318 and avoiding exposure of the virally infected area of skin to bright light 320. The method 310 includes also the steps of allowing the area of skin to become photosensitive 314, rinsing the photosensitive area of skin 322, and illuminating a beam of visible light onto the photosensitive skin 316 to produce a photodynamic reaction substantially reducing the amount of viral infection to the skin.

In operation, the photosensitizing agent is applied 12, 112, 212, 312 once and the beam of light is illuminated 16, 116, 216, 316 once per treatment site. The photosensitization of the virally infected skin 14, 114, 214, 314 following application of the photosensitizing agent to such skin 12, 112, 212, 312 occurs through the metabolic conversion of the ALA to PplX. The PplX accumulates in the skin to which the photosensitizing agent has been applied 12, 112, 212, 312. When exposed to the visible light 16, 116, 216, 316 of appropriate wavelength and energy, the accumulated PplX produces the photodynamic reaction. The photodynamic reaction is a cytotoxic process dependent upon the simultaneous presence of light and oxygen. The method 10, 110, 210, 310 substantially reduces the amount of viral infection to the skin in about eight weeks.

The method for treating dermatological viral infections 10, 110, 210, 310 is not associated with any substantial discomfort. The method 10, 110, 210, 310 is also for people of all ages. The method 10, 110, 210, 310 also, if there are many growths, does not require multiple treatment sessions until the growths are gone. The method 10, 110, 210, 310 also eliminates the need to wait for the growths to go away on their own. The method 10, 110, 210, 310 also minimizes the possibility for a person's skin to get infected again with a particular virus.

The method 10, 110, 210, 310 has been described in an illustrative manner. It is to be understood that the terminology that has been used is intended to be in the nature of words of description rather than of limitation. Many modifications and variations of the method 10, 110, 210, 310 are possible in light of the above teachings. Therefore, within the scope of the appended claims, the method 10, 110, 210, 310 may be practiced other than as specifically described.

What is claimed is:

1. A method for treating a dermatological viral infection comprising the steps of:
applying a photosensitizing agent to virally infected skin;
allowing the virally infected skin to become photosensitive; and
illuminating a beam of visible light onto the photosensitive skin to produce a photodynamic reaction substantially reducing the amount of viral infection to the skin.

2. The method of claim 1, wherein the dermatological virus includes warts.

3. The method of claim 1, wherein the dermatological virus includes any virus belonging to the poxvirus family of viruses.

4. The method of claim 3, wherein the dermatological virus includes molluscum contagiosum.

5. The method of claim 1, wherein the virally infected skin to be treated is clean and dry prior to the step of applying the photosensitizing agent to the virally infected skin.

6. The method of claim 1, wherein said step of applying the photosensitizing agent is performed topically.

7. The method of claim 6, wherein said step of topically applying the photosensitizing agent is performed substantially directly and uniformly to only individual areas of the virally infected skin.

8. The method of claim 1, wherein the photosensitizing agent includes aminolevulinic acid HCL.

9. The method of claim 8, wherein the photosensitizing agent is Levulan® Kerastick® Topical Solution.

10. The method of claim 1, wherein the time period for performing the step of allowing the skin to become photosensitive is about fourteen to about eighteen hours.

11. The method of claim 1, wherein the beam of visible light is of substantially low intensity such that the beam of visible light does not substantially heat the photosensitive skin.

12. The method of claim 11, wherein the beam of visible light is illuminated at an intensity of about 6 to about 10.9 J/cm² for about seventeen minutes.

13. The method of claim 12, wherein the beam of visible light is illuminated at an intensity of about 10 J/cm² for about 16 minutes and 40 seconds.

14. The method of claim 1, wherein the photosensitive skin is located between about two inches and about four inches from the source of the beam of visible light.

15. The method of claim 1, wherein the source of the beam of visible light is a laser.

16. The method of claim 1, wherein the beam of visible light is blue.

17. The method of claim 16, wherein the wavelength of the beam of blue light is between about 400 nm and about 450 nm.

18. The method of claim 17, wherein the beam of blue light is illuminated using BLU-U™ Blue Light Photodynamic Therapy Illuminator.

19. A method for treating a dermatological viral infection comprising:
applying a photosensitizing agent to virally infected skin;
keeping the virally infected skin dry;
avoiding exposure of the virally infected skin to bright light;
allowing the virally infected skin to become photosensitive; and
illuminating a beam of visible light onto the photosensitive skin to produce a photodynamic reaction substantially reducing the amount of viral infection to the skin.

20. A method for treating a dermatological viral infection comprising the steps of:
applying a photosensitizing agent to virally infected skin;
allowing the virally infected skin to become photosensitive;
rinsing the photosensitive skin; and
illuminating a beam of visible light onto the photosensitive skin to produce a photodynamic reaction substantially reducing the amount of viral infection to the skin.

21. A method for treating a dermatological viral infection comprising the steps of:
applying a photosensitizing agent to virally infected skin;
keeping the virally infected skin dry;
avoiding exposure of the virally infected skin to bright light;
allowing the virally infected skin to become photosensitive;
rinsing the photosensitive skin; and
illuminating a beam of visible light onto the photosensitive skin to produce a photodynamic reaction substantially reducing the amount of viral infection to the skin.
